(19) United States
(12) Patent Application Publication
(10) Pub. No.: US 2007/0021364 A1
(43) Pub. Date: Jan. 25, 2007

(54) Methods for genetic immunization

(76) Inventors: Hans Herweijer, Brooklyn, WI (US); Jon A. Wolff, Madison, WI (US); Mary Kay Bates, Madison, WI (US); Aaron G. Loomis, Prairie du Sac, WI (US); Vladimir Trubetskoy, Middleton, WI (US)

Correspondence Address: MIRUS CORPORATION, 505 SOUTH ROSA RD, MADISON, WI, 53719, US

(21) Appl. No.: 11/437,933

(22) Filed: May 19, 2006

Related U.S. Application Data
(63) Continuation-in-part of application No. 09/992,957, filed on Nov. 13, 2001, now abandoned.
(63) Continuation-in-part of application No. 10/600,098, filed on Jun. 20, 2003.
Application No. 10/600,098 is a division of application No. 09/447,966, filed on Nov. 23, 1999, now Pat. No. 6,627,616.
Application No. 09/447,966 is a continuation-in-part of application No. 09/391,260, filed on Sep. 7, 1999, now abandoned.
Application No. 09/391,260 is a division of application No. 08/975,573, filed on Nov. 21, 1997, now Pat. No. 6,265,387.
Application No. 08/975,573 is a continuation of application No. 08/571,536, filed on Dec. 13, 1995, now abandoned.
(60) Provisional application No. 60/248,275, filed on Nov. 14, 2000.

(51) Int. Cl. A61K 48/00; C12P 21/08; C07K 16/18; C12N 5/06

(52) U.S. Cl. 514/44; 435/69.1; 435/320.1; 435/325; 530/388.1; 536/23.53

(57) ABSTRACT

The present invention relates to methods for delivering a genetic immunogen, comprising a nucleic acid capable of expressing an antigen, optionally complexed with a polymer. The nucleic acid is delivered to the host via hydrodynamic intravascular injection resulting in expression of an encoded antigen in extravascular cells and induction of an antigen-specific immune response.

### CROSS-REFERENCE TO RELATED APPLICATIONS

This application is a continuation-in-part of application Ser. No. 09/992,957, filed Nov. 14, 2001, and a continuation-in-part of application Ser. No. 10/600,098, filed Jun. 20, 2003, which is divisional of application Ser. No. 09/447,966 filed Nov. 23, 1999, now U.S. Pat. No. 6,627,616, which is a continuation-in-part of application Ser. No. 09/391,260, filed on Sep. 7, 1999, abandoned, which is a divisional of application Ser. No. 08/975,573, filed no Nov. 21, 1997, now U.S. Pat. No. 6,267,387, which is a continuation of application Ser. No. 08/571,536, filed on Dec. 13, 1995, now abandoned. Application Ser. No. 09/992,957 claims the benefit of U.S. Provisional Application No. 60/248,275, filed Nov. 14, 2000.

### FIELD OF THE INVENTION

The present invention relates to compositions and methods for transferring nucleic acids into cells in vivo for the purpose of eliciting an immune response. In preferred embodiments, the compositions include intravascular delivery systems providing high transfection efficiency; the compositions further include delivery systems providing nucleic acid transfer complexes that transfect cells with high efficiency; and methods for detection of an immune response following genetic immunization.

### BACKGROUND OF THE INVENTION

Vaccination, or immunization, stimulates the immune system of an animal. The immune system of vertebrates consists of several interacting components. Two of the most important components are the humoral and cellular responses. Antibody molecules, immunoglobulins, are the effectors of the humoral immune response and are secreted by special B lymphoid cells, called B cells. Antibodies can bind to and inactivate antigen directly (neutralizing antibodies) or activate other cells of the immune system to destroy the antigen. The cellular immune response is mediated by a special class of lymphoid cells, the cytotoxic T cells or cytotoxic T lymphocytes (CTLs). These cells respond to peptide fragments which appear on the surface of a target cell bound to major histocompatibility complex (MHC) proteins. The cellular immune system is constantly monitoring the proteins produced in all cells in the body in order to eliminate cells producing foreign antigens. Humoral immunity is directed mainly at antigens which are exogenous to the animal whereas the cellular system responds to antigens which are actively synthesized within the animal. Cellular immunity complements the humoral system by eliminating the infected host cells. A vaccination can elicit a humoral immune response, a cellular (cytotoxic) immune response, or both.

The development of vaccines is frequently heralded as one of the most important medical breakthroughs. Prevention of disease has increased human life expectancy, lowered healthcare costs, and enhanced quality of life. Classically, vaccines have consisted of the antigen itself delivered to the animal. The antigen can be in the form of an attenuated, killed or inactivated bacteria or virus, or as purified native or recombinant polypeptide. Thus, the development of new vaccines requires identification, isolation or purification of the appropriate infectious agent or antigen. If the infectious agent is to be used, it must be sufficiently inactivated to safely activate the immune system without causing illness. Genetic vaccines provide solutions to these problems. Because the delivered component is a nucleic acid sequence, genetic vaccines are more readily adapted to emerging and mutating infectious diseases and are easier to produce and store. With a genetic vaccine, a gene encoding the antigen is introduced into the host. Following delivery of the gene into host cells, the gene is expressed and the resultant peptide, the antigen, is presented to the immune system.

Since each individual genetic vaccine requires just the coding sequence for the antigen, many different vaccines can be produced and tested for each microbe. It is even feasible to generate a shot-gun library for a given microbe, vaccinate an appropriate animal model, and determine which clones result in the greatest immunity (either humoral or cellular). Alternatively, the expression of multiple epitopes allows genetic vaccines to better cover the variability in antigen presentation that exists in the population due to major histocompatibility complex (MHC) polymorphism. By expressing antigens in vivo one avoids the use of killed or attenuated microbes. Also, it is possible to create vaccines for peptides that previously could not be produced or isolated. Since the full cellular biochemical machinery is available, antigens that are heavily modified can be used efficiently.

Immune responses following genetic vaccination/immunization have been reviewed in detail (Donnelly J J et al. 1997; Pardoll DM et al. 1995). Genetic vaccines elicit both strong humoral and cellular immune responses. In contrast, conventional subunit (purified antigen) vaccines are typically skewed toward humoral responses. Attenuated microbe vaccines, which typically provide better immunity, typically elicit stronger cytotoxic T cell responses. Genetic vaccines are therefore more likely to provide better immunity than subunit vaccines while being safer than attenuated microbe vaccines.

Genetic vaccines have proven effective in eliciting immune responses against a wide variety of microbes. Protection in animal models has been demonstrated for influenza virus, malaria, bovine herpes virus, rabies virus, papilloma virus, herpes simplex virus, mycoplasma, lymphocytic choriomeningitis and others. The art has established that direct injection of pDNA into muscle in mice is an efficient, reliable method for genetic vaccine delivery. However, gene transfer following intramuscular injection of pDNA is less efficient in larger rodents and primates. Human genetic vaccine trials have corroborated these earlier gene transfer and expression studies, by finding the need to inject large amounts of pDNA in human muscles to obtain good immune responses. Complexing pDNA with cationic liposomes (lipoplexes) has been attempted to enhance the efficiency of intramuscular and intranasal delivery.

The completion of sequencing of the human genome has identified a very large number of open reading frames with no information on the function of the protein. For identification, purification and functional research, specific antibodies remain among the most useful tools. In addition, fully humanized antibodies can be used successfully as therapeutic agents. The sequencing of other genomes will only contribute to the need for new antibodies. Antibodies (polyclonal and monoclonal) also form the basis for many diagnostic assays. Antibody-based assay are important in the detection and study of microbial agents and infected hosts. Novel assays and antibodies are constantly required following the detection of previously unknown microbial agents. Obtaining sufficient agent material for immunization can be difficult and can pose significant biohazards. Immunization with complete agents will typically yield antibodies specific for a few immunodominant epitopes, thus not allowing for detailed biological investigations. Genetic immunization techniques overcome these issues.

### SUMMARY OF THE INVENTION

The present invention provides methods for delivering an antigen to a vertebrate in vivo comprising: introducing a polynucleotide coding for the antigen into a vessel in the vertebrate whereby the polynucleotide is delivered into the interior of a cell in the vertebrate and the antigen is expressed and presented to the immune system of the vertebrate. The polynucleotide codes for an immunogenic peptide that is expressed by the transfected cells thereby generating an antigen-specific immune response. Generation of the immune response may immunize the vertebrate. Generation of the immune response may also provide polyclonal antibodies, monoclonal antibodies, or immune cells of interest. The methods can be used for the production of antibodies in a vertebrate, to provide a vaccine, or to provide a therapeutic response, such as to cancer or infection.

In a preferred embodiment, methods are described for vaccinating, or immunizing, a vertebrate, comprising: forming an expressible polynucleotide encoding an antigen and, injecting the polynucleotide into a vessel in the vertebrate thus delivering the polynucleotide to a cell in the vertebrate wherein the translation product of the polynucleotide, the antigen, is formed by the cell thereby eliciting an immune response against the antigen. The polynucleotide is injected into the vessel using a volume and rate sufficient to elevate intravascular pressure, increase permeability of tissue vasculature to the polynucleotide and deliver the polynucleotide into extravascular cells in the tissue. The antigen may be delivered to a variety of cell types using the methods of the present invention, including, but not limited to, liver cells, spleen cells, heart cells, lymph node cells, skeletal muscle cells, lung cells, thymus cells, kidney cells, skin cells, pancreas cells, intestinal cells, mucosal cells, antigen presenting cells, T cells, B cells, natural killer (NK) cells, dendritic cells, and macrophages. The antigen may be secreted by the cell, or it may be presented by the cell in the context of a major histocompatibility complex. The method may be used to selectively elicit a humoral immune response, a cellular immune response, or a mixture of these.

In a preferred embodiment, the antigen-encoding polynucleotide is injected by hydrodynamic intravascular delivery into a vessel in a vertebrate. Hydrodynamic intravascular delivery comprises rapidly injecting a relatively large volume of a pharmaceutically acceptable carrier into an efferent or afferent vessel of a tissue in which the target cell resides, resulting in transiently elevated intravascular pressure, increased vessel permeability to nucleic acid, and increased extravascular volume in the target tissue. In another preferred embodiment the polynucleotide is introduced into the tail vein of a rodent by hydrodynamic tail vein injection. In yet another preferred embodiment, the polynucleotide is injected into a vessel in a limb of the vertebrate.

In a preferred embodiment the polynucleotide may be introduced into the vertebrate using an injectable carrier alone. The carrier preferably is isotonic, hypotonic, or weakly hypertonic, such as provided by a sucrose, saline, or Ringer's solution. The polynucleotide may also be associated with or complexed with other compounds prior to injection of the polynucleotide into the vertebrate.

In a preferred embodiment the transferred polynucleotide expresses an antigen that induces an antigen-specific immune response. The antigen-specific immune response results in the formation of antigen-specific antibodies. The antigen-specific antibodies may be obtained and purified from the blood of the host. In a preferred embodiment B cells that produce antigen-specific antibodies may be obtained from the host. The B cells may be fused with myeloma cells to create monoclonal antibody producing cells. In another preferred embodiment the genetic immunization results in the induction of an antigen-specific cellular immune response. The immune response may result in the induction of T cells or NK cells or both.

In a preferred embodiment, the polynucleotide encodes an antigen of an intracellular infectious agent or an antigen encoded by a cellular gene. An intracellular infectious agent may be a viral pathogen, a bacterial pathogen, a fungal pathogen, a protozoan, or other intracellular pathogen. A cellular gene may be a gene that is expressed in a cancer or tumor cell. The antigen can also be from a protein of known or unknown function. The antigen is expressed in a cell and presented in the context of the MHC complex thereby stimulating a cellular immune response. The immune response may stimulate cytotoxic T cells that are capable of destroying infected or cancer/tumor cells. In another preferred embodiment, the polynucleotide encodes an extracellular antigen. The antigen may be expressed from the polynucleotide inside the cell and secreted by the cell.

In a preferred embodiment, the polynucleotide may be co-delivered with another agent to modulate or induce an immune reaction. The agent may be a polynucleotide, drug, protein, or other compound known to enhance, alter, augment, or inhibit one or more types of immune responses.

In a preferred embodiment, polynucleotides may be delivered to extravascular limb cells to provide for expression of a peptide or protein antigen. We show that intravenous administration of a polynucleotide-containing solution results in delivery of the polynucleotide to nonvascular parenchymal cells, including skeletal muscle cells, expression of a gene encoded by the polynucleotide in the cells, and induction of an immune response in the mammal. The polynucleotide can encode a peptide or protein antigen to generate an immune response in the animal. The described process can be used for the production of antibodies in a mammal, to provide a vaccine, or to provide a therapeutic response, such as to cancer or infection.

In a preferred embodiment, an immunizing polynucleotide may be transfected into a cell in vitro to produce the antigen. The antigen can subsequently be used for determining the presence, amount, and affinity of antibodies directed against it.

Further objects, features, and advantages of the invention will be apparent from the following detailed description when taken in conjunction with the accompanying drawings.

### BRIEF DESCRIPTION OF THE DRAWINGS

FIG. 1. Immunohistochemical staining of ICR mouse skeletal muscle with antisera from mice genetically immunized with a polynucleotide encoding human dystrophin. The left panel shows muscle stained with the anti-human dystrophin antisera using a labeled anti-mouse IgG secondary antibody for fluorescence detection. The right panel shows mouse muscle stained with human-specific anti-human dystrophin monoclonal antibody.

FIG. 2. Western blots illustrating presence of antibodies to mammalian proteins in mice immunized with polynucleotides encoding human CD4 or canine dystrophin. Panel A shows detection of antigen using antisera from mice injected with CD4 encoding polynucleotide (predicted size 46 kD). Panel B shows detection of antigen using antisera from mice injected with dystrophin encoding polynucleotide (predicted size 425 kD). Each set of two lanes (− and +) represents serum from an individual mouse (−lane =cell extract lacking antigen; +lane =cell extract containing antigen).

FIG. 3. Western blot showing induction of luciferase-specific antibodies in rats following intravascular genetic immunization. The blot contains cell extracts for COS7 cells either expressing a control protein (−lanes) or luciferase (+lanes). Rat antisera were used at a 1:100 dilution. Secondary anti-rat HRP antibody was used at a 1:5000 dilution.

FIG. 4. Graph illustrating immune response in mice immunized with different expression vectors with or without booster injection. Legend indicates expression vector used to drive luciferase expression. CMV=cytomegalovirus promoter vector. UbC=ubiquitin C promoter vector. The mice were immunized with plasmid DNA vectors expressing luciferase under transcriptional control of the CMV or the ubiquitin C promoter.

FIG. 5. Anti-luciferase antibody titers in mice genetically immunized via delivery by hydrodynamic tail vein injection, direct intramuscular injection, and intravascular DNA/PEI/PAA particle injection.

FIG. 6. Graph illustrating antibody production against luciferase protein following genetic immunization of rabbits via limb vein injection. A. Graph illustrating the time course of antibody expression detected via ELISA. B. Western blot using serum from immunized rabbit. The blot contained cell extracts for COS7 cells either expressing a control protein (− lane) or luciferase (+ lane). Rabbit antisera were used at a 1:100 dilution. Secondary anti-rat HRP antibody was used at a 1:5000 dilution.

FIG. 7. Western blot illustrating antibody detection using extracts from in vitro transfected cells. Lane 1, 5 ng recombinant P-galactosidase protein; Lane 2, 1 ng recombinant luciferase protein; Lane 3, 5 ng recombinant luciferase protein; Lanes 4-5, COS-7 cells transfected with pCI-LacZ (4) or pCILuc (5); Lanes 6-7, HEK 293 cells transfected with pCI-LacZ (6) or pCI-Luc (7); Lane 8-9, Hepa-lclc7 cells transfected with pCI-LacZ (8) or pCI-Luc.

FIG. 8. Immunohistochemical staining of HeLa cells probed with monoclonal antibody sera generated via intravascular genetic immunization of mice. Panel A shows Transduction Laboratories control anti-Ki67 monoclonal antibody (used at 1 μg/ml) generated via classical protein purification and injection. Panels B-F show five different culture supernatants from hybridoma fusions generated from mice immunized against Ki67 using intravascular delivery of polynucleotide. Secondary antibody was Cy3-labeled anti-mouse IgG (H+L) F(ab′)2 fragment.

FIG. 9. Western blot illustrating enhanced immune response from codon optimized genetic immunization vectors. Duplicate transfected HeLa cell lysates were run in two SDS polyacrylamide gels and each gel was transferred to Hybond-P (Amersham Biosciences). One blot was probed with chicken anti-NS 1 IgY and the other was probed with rabbit anti-NS2 serum. Blots were developed using appropriately conjugated secondary antibodies and chemiluminescent detection.

### DETAILED DESCRIPTION OF THE INVENTION

We describe methods to elicit an antigen-specific immune response in a vertebrate via genetic immunization. Genetic immunization comprises delivering to a cell in vivo a polynucleotide encoding one or more antigens against which an immune response is to be generated. For genetic immunization to generate an antigen-specific immune response, the gene of interest must be delivered to host cells and expressed. The described methods comprise delivery systems for polynucleotides in vivo. The in vivo delivery and expression of the polynucleotide results in an immune response directed against an encoded antigen. A polynucleotide encoding an antigen (immunogen or immunogenic polypeptide) of interest is injected into a vessel of a vertebrate in a volume and at a rate that facilitates increasing permeability of vasculature in the vertebrate and delivery of the polynucleotide to an extravascular cell. The delivered polynucleotide is then expressed, producing the antigen in vivo.

The immune response may result in the formation of antigen-specific antibodies, the induction of an antigen-specific cellular immune response, or other forms of immune response. The immune response may be directed against proteins associated with conditions, infections, diseases or disorders such as pathogen antigens or antigens associated with cancer cells.

The polynucleotide may be delivered to a cell in vivo to elicit a cell mediated immune response. The polynucleotide may also be delivered to a cell in vivo to elicit a humoral response. Cell mediated immunity is mediated by cells or the products they produce, such as cytokines, rather than by antibody production. It includes, but is not limited to, delayed type hypersensitivity and cytotoxic T cells (cytotoxic T lymphocytes, CTL). The term humoral immunity relates to an immune response mediated by antibodies and the cells involved in the production of antibodies and subsequent activity with the antigen. Cell mediated and humoral immunity are often induced simultaneously and influence each other. Since the immune systems of all vertebrates operate similarly, the applications described can be implemented in all vertebrate systems, comprising mammalian and avian species, as well as fish.

Genetic vaccination may be used for a number of purposes, including, but not limited to, generation of antibodies or immune cells for research, analytical, diagnostic or therapeutic purposes, to immunize an animal against subsequent infection, and to boost an animals immune response against a current condition, and to study immune response. Antibodies can be generated against antigens associated with infectious agents, allergens, cancer cell, proteins of interest to biological researchers, and the like. Further, the antigen can be from a polypeptide of known or unknown fimction.

The genetic vaccine is injected into a vessel in a vertebrate and delivered to cells of the vertebrate. The sequence encoded by an expression cassette is expressed and the resultant immunogenic peptide is produced. An immune response is then induced by the vertebrate against the immunogenic peptide. The immunogenic peptide refers to peptides or proteins encoded by gene constructs of the present invention which act as target antigens for an immune response. The immune response can be directed against proteins associated with conditions, infections, diseases or disorders such as allergens, pathogen antigens, antigens associated with cancer cells, cells involved in autoimmune diseases or other proteins of interest. The vaccinated individual may be immunized prophylactically or therapeutically to prevent or treat conditions, infections, diseases or disorders. A vaccinated animal may also be used to generate antibodies or immune cells. The immunogenic peptide shares at least one epitope with a protein from the allergen, pathogen, protein or cell-type such as an infected cell, a cancer cell or a cell involved in autoimmune disease against which immunization is desired. The immune response directed against the immunogenic peptide can protect the individual against disease or infection, treat the individual for the specific infection or disease with which the polypeptide from the allergen, pathogen or undesirable protein or cell-type is associated. The immunogen does not need to be identical to the protein against which an immune response is desired. Rather, the immunogenic target polypeptide must be capable of inducing an immune response that cross reacts with the protein against which the immune response is desired.

Genetic immunization may be used to provide a method to treat latent viral infections. Several viruses, such as Hepatitis B, HIV and Herpes viruses, can establish latent infections in which the virus is maintained intracellularly in an inactive or partially active form. By inducing a cellular immune response against such viral infections, the infected cells can be targeted and eliminated. Chronic pathogen infections or poorly immunogenic infections may be similarly treated. There are numerous examples of pathogens which replicate slowly and spread directly from cell to cell. CTL directed killing of the infected cells can eliminate or slow the disease. The genetic immunization can be used to generate an immune response against infectious pathogens selected from the list comprising: immunodeficiency virus, human hepatitis A virus, human hepatitis B virus, human hepatitis C virus, influenza virus, smallpox (variola) virus, human herpes virus (type I through VIII), Bacillus, Bordetella, Borrelia, Brucella, Chlamydia, Clostridium, Corynebacterium, Escherichia, Haemophilus, Legionella, Listeria, Mycobacterium, Mycoplasma, Neisseria, Rickettsia, Salmonella, Staphylococcus, Streptococcus, Treponema, Vibrio, Yersinia, fungal pathogens, and pathogenic protozoans.

Genetic immunization can also be used to treat established diseases, such as but not limited to: cancer, tumor, and autoimmune disease. A number of tumor antigens which are recognized by T lymphocytes of the immune system have been identified and are considered as potential vaccine candidates. Therapeutic vaccination to mount a cellular immune response to a protein specific to the malignant state, be it an activated oncogene, a fetal antigen or an activation marker, may result in the elimination of these cells.

A cancer vaccine is a method of treating the disease involving administration of one or more characteristic antigens of the cancer often in combination with factors that boost immune function. This induces the patient's immune system to attack and eliminate the cancerous cells. Unlike traditional vaccines for infectious diseases, cancer vaccines are not typically given to prevent the initial development of cancer. Instead, cancer vaccines are a method of treating cancer that has already occurred and are given to patients already diagnosed with cancer. As a cancer treatment method, the goal of cancer vaccines is to reduce or eliminate tumor or cancerous cells from the body. Cancer vaccines can be given after or in conjunction with more traditional cancer treatments, such as chemotherapy, radiation, or surgery. Cancer vaccines can also be given with the aim of suppressing the recurrence of the cancer.

Genetic cancer vaccines contain genetic material encoding an antigen associated with a tumor cell. Some antigens are unique to a cancer type, some are unique to an individual tumor, while a very few are found in more than one cancer type. For example, vaccines against telomerase and survivin, two proteins produced by many cancers, have been developed, raising hopes for the development of a universal cancer vaccine.

The immune response may be aimed at obtaining antibodies or immune cells specific for the antigen, for example B cells producing antibodies. These immune cells or immune cell products may be used for analytical or therapeutic purposes. As demonstrated by the data herein, the genetic immunization methods of the present invention provides substantially higher immune response efficiencies than other available systems. Genetically immunized animals may be used to produce polyclonal or monoclonal antibodies. The means for isolating, preparing and characterizing antibodies are well known in the art.

Cells of the immune system include antigen presenting cells, which process antigens and present them to other immune cells, including helper T cells, T-effector lymphocytes, natural killer cells, polymorphonuclear leukocytes, macrophages, dendritic cells, basophils, neutrophils, eosinophils, monocytes.

Genetic immunizations have several advantages over conventional (polypeptide) immunizations. By introducing a plasmid DNA expression vector into the host, the antigen is synthesized in situ. This methodology is superior to using polypeptide antigens, since: a) not all polypeptides are easily produced, purified, or synthesized, b) natural epitopes are more likely to be presented to the immune system than peptides, c) unlike polypeptides obtained from bacteria or synthesized, in vivo produced peptides are more likely to have native modifications (e.g., glycosylation), and d) production of a genetic vaccine is faster and cheaper. Because of these advantages, it is easier to screen multiple epitopes with genetic immunization.

Any peptide-based antigen which is a candidate for an immune response, whether humoral or cellular, can be used in its polynucleotide form. The genetic immunization may comprise a single injection of polynucleotide, a prime injection. Alternatively, the genetic immunization may comprise multiple injections of the polynucleotide, an initial prime injection and one or more subsequent boost (or booster) injections. Boosting can be repeated until a desired level of immune response is achieved.

For genetic immunization, the transferred polynucleotide encodes a polypeptide which is expressed and induces a desired immune response. The expressed antigen may be secreted by the cell or be presented by the cell in the context of the major histocompatibility complex, thereby eliciting an immune response. The cell may be a professional antigen presenting cell (APC) or a non-professional antigen presenting cell (non-APC). The antigen may be expressed in a non-APC and then taken up by an APC, in a process termed cross-priming (Larrson M et al. 2001; Clotilde T et al. 2001; Doe B et al. 1996). For example, the expressed antigen may leak from the transfected cell and be taken up by an APC (e.g., in the draining lymph nodes). The antigen may be released in the context of SOS signals, heat shock proteins, etc., and taken up by an APC. The APC then presents the antigen to other immune cells. The method may be used to selectively elicit a humoral immune response (B cell mediated), a cellular immune response (T-cell mediated), or a mixture of these. An antigen refers to a molecule, such as a peptide, capable of eliciting an immune response.

An antigen also refers to any agent that is recognized by an antibody or antibodies. The term antigenic determinant, or epitope, refers to a site on an antigenic molecule which binds to an antibody or specific receptor site on the sensitized lymphocyte. Thus, a single peptide, or antigen, can possess one or more antigenic determinants. The term immunogen refers to any agent that can elicit an immunological response in an animal. In many cases, antigens are also immunogens, thus the term antigen is often used interchangeably with the term immunogen. These terms may be used to refer to an individual macromolecule or to a homogeneous or heterogeneous population of antigenic molecules. The antigenic moiety can also be a subunit of a protein, peptide, chimeric polypeptide, recombinant polypeptide or similar product. A chimeric polypeptide comprises two or more peptide sequences derived from different genes but expressed as a single polypeptide sequence. For genetic immunization, the antigen or immunogen is a polypeptide expressed from a delivered polynucleotide. The genetic immunization may elicit an immune response against a single antigen or against a plurality of antigens.

A hapten is a substance that reacts selectively with appropriate antibodies or T cells but the hapten itself is usually not immunogenic. Most haptens are small molecules or small parts of large molecules, but some macromolecules can also fimction as haptens.

Immunogenic peptide or immunogen is meant to refer to an antigen that is a target for an immune response and against which an immune response can be elicited. The immunogenic peptide shares at least one epitope with a protein against which immunization is desired. In one application, the immune response is directed at proteins associated with conditions, infections, diseases or disorders such as allergens, pathogen antigens, antigens associated with cancer cells or cells involved in autoimmune diseases. In another application, the antigen-directed immune response is applied to biological studies, the generation of cellular or humoral immune response products (e.g., CTL clones, B cells, plasma cells, antibodies), or derivatives thereof (e.g., monoclonal antibodies). The immunogenic antigen is encoded by the coding sequence of a genetic construct called an expression vector.

The term antibody encompasses whole immunoglobulin of any class, chimeric antibodies, hybrid antibodies with dual or multiple antigen specificities and fragments including hybrid fragments. Also included within the meaning of antibody are conjugates of such fragments, and so-called antigen binding proteins (single chain antibodies) as described, for example, in U.S. Pat. No. 4,704,692. Alternatively, the encoded antibodies can be anti-idiotypic antibodies (antibodies that bind other antibodies) as described, for example, in U.S. Pat. No. 4,699,880.

The described immunization system comprises a hydrodynamic intravascular injection method for delivery of the polynucleotide. Hydrodynamic intravascular delivery comprises rapidly injecting a relatively large volume of a pharmaceutically acceptable carrier into an efferent or afferent vessel of a tissue in which the target cell resides, resulting in transiently elevated intravascular pressure, increased vessel permeability to nucleic acid, and increased extravascular volume in the target tissue. Hydrodynamic intravascular injection has been shown to be reliable and efficient in a number of animal species, including rodent, dog, pig, and non-human primate. Because the method is readily adapted to use in rats, dogs, and nonhuman primates, it is expected that the method is also readily adapted to use in other animals, including humans. The described immunization system has also been shown to be reliable and efficient. While not every peptide antigen is strongly immunogenic, and some peptides may not elicit an immune response regardless of the method of delivery, we have observed consistent immune induction for antigens which do elicit an immune response. Using the described genetic immunization method, it is possible to obtain an immune response in more than 50%, more than 60%, more than 70%, more than 80%, or more than 90% of individual animals injected.

Vessels comprise internal hollow tubular structures connected to a tissue or organ within the body of an animal, including a mammal. Bodily fluid flows to or from the body part within the lumen of the tubular structure. Examples of bodily fluid include blood, lymphatic fluid, or bile. Vessels comprise: arteries, arterioles, capillaries, venules, sinusoids, veins, lymphatics, and bile ducts. As used herein, the term vessel does not encompass the intestines, uterus, esophagus, stomach or bladder. Afferent vessels are directed toward the organ or tissue and in which fluid flows toward the organ or tissue under normal physiological conditions. Conversely, efferent vessels are directed away from the organ or tissue and in which fluid flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood toward the liver. A vascular network consists of the directly connecting vessels supplying and/or draining fluid in a target organ or tissue.

The choice of injection volume and rate are dependent upon: the size of the animal, the size of the vessel into with the solution is injected, the size and or volume of the target tissue, the bed volume of the target tissue vasculature, and the nature of the target tissue and vessels supplying the target tissue. For example, delivery to liver may require less volume because of the porous nature of the liver vasculature. The precise volume and rate of injection into a particular vessel, for delivery to a particular target tissue, may be determined empirically. Larger injection volumes or higher injection rates or both are typically required for larger vessels, target sizes, etc. For example, efficient delivery to mouse liver may require injection of as little as 1 ml or less (animal weight˜25 g). In comparison, efficient delivery to dog or nonhuman primate limb muscle may require as much as 60-500 ml or more (animal weight 3-14 kg). Injection rates can vary from 0.5 ml/sec or lower to 4 ml/sec or higher, depending on animal size, vessel size, etc. Occlusion of vessels, by balloon catheters, clamps, cuffs, natural occlusion, or other means can limit or define the vascular network size or target area.

Injecting into a vessel an appropriate volume at an appropriate rate increases permeability of the vessel to the injection solution and the molecules or complexes therein and increases the volume of extravascular fluid in the target tissue. Permeability can be further increased by injecting the polynucleotide while occluding outflow of fluid (both bodily fluid and injection solution) from the tissue or local vascular network. Permeability is defined herein as the propensity for macromolecules such as nucleic acids to move through vessel walls and enter the extravascular space. One measure of permeability is the rate at which macromolecules move through the vessel wall and out of the vessel. Another measure of permeability is the lack of force that resists the movement through the vessel wall and out of the vessel. Vessels contain elements that prevent macromolecules from leaving the intravascular space (internal cavity of the vessel). These elements include endothelial cells and connective material (e.g., collagen). Increased permeability indicates that there are fewer of these elements that can block the egress of macromolecules or that the spaces between these elements are larger and more numerous or both. In this context, increased permeability enables a higher percentage of macromolecules being delivered to leave the intravascular space, while low permeability indicates that a low percentage of the macromolecules will leave the intravascular space.

Vasculature permeability may be further increased by increasing the osmotic pressure within the vessel. Typically, hypertonic solutions containing salts such as sodium chloride, sugars or polyols such as mannitol are used. Hypertonic means that the osmolality of the injection solution is greater than physiologic osmolality. Isotonic means that the osmolality of the injection solution is the same as the physiological osmolality (i.e., the tonicity or osmotic pressure of the solution is similar to that of blood). Hypertonic solutions have increased tonicity and osmotic pressure compared to the osmotic pressure of blood and cause cells to shrink.

The permeability of the blood vessel can also be further increased by administering a biologically-active molecule such as a protein or a simple chemical such as histamine that increases the permeability of the vessel by causing a change in function, activity, or shape of cells within the vessel wall such as the endothelial or smooth muscle cells. Typically, biologically active molecules that affect permeability interact with a specific receptor or enzyme or protein within the vascular cell to change the vessel's permeability. Biologically active molecules include vascular permeability factor (VPF), which is also known as vascular endothelial growth factor (VEGF). Another type of biologically active molecule can also increase permeability by changing the extracellular connective material. For example, an enzyme could digest the extracellular material and increase the number and size of the holes of the connective material. Other biologically active molecules that may alter the permeability include calcium channel blockers (e.g., verapamil, nicardipine, diltiazem), beta-blockers (e.g., lisinopril), phorbol esters (e.g., PKC), ethylenediaminetetraacetic acid (EDTA), adenosine, papaverine, atropine, and nifedipine.

The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyl-uracil, dihydrouracil, inosine, N6-isopentenyladenine, 1-methyladenine, 1-methyl-pseudouracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, β-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations of DNA, RNA and other natural and synthetic nucleotides.

DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 19-25 base pairs and having a nucleotide sequence identical or nearly identical to an expressed target gene or RNA within the cell. Interference may result in suppression of expression. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Polynucleotides may contain an expression cassette coded to express a whole or partial protein, or RNA. An expression cassette refers to a natural or recombinantly produced polynucleotide that is capable of expressing a sequence. The term recombinant as used herein refers to a polynucleotide molecule that is comprised of segments of polynucleotide joined together by means of molecular biological techniques. The cassette contains the coding region of the gene of interest along with any other sequences that affect expression of the sequence of interest. An expression cassette typically includes a promoter (allowing transcription initiation) and a transcribed sequence. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences. The regulatory sequences of the expression cassette may be selected to be appropriate for the target cell and host. The choice of regulatory sequences in the expression cassette may also depend on the duration of expression desired. For some applications, it is desirable that the antigen be expressed for a short period of time. For other applications, longer term expression may be desired.

The polynucleotide may contain sequences that do not serve a specific function in the target cell but are used in the generation of the polynucleotide. Such sequences include, but are not limited to, sequences required for replication or selection of the polynucleotide in a host organism.

The term gene generally refers to a polynucleotide sequence that comprises coding sequences necessary for the production of a therapeutic polynucleotide (e.g., ribozyme) or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′and 3′ends for a distance of about 1 kb or more on either end such that the gene corresponds to the length of the full-length mRNA. The sequences that are located 5′of the coding region and which are present on the mRNA are referred to as 5′untranslated sequences. The sequences that are located 3′or downstream of the coding region and which are present on the mRNA are referred to as 3′untranslated sequences. The term gene encompasses both cDNA and genomic forms of a gene. A genomic form or clone of an eukaryotic gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the mature RNA transcript.

The messenger RNA (mRNA) functions during translation to specify the sequence or order of amino acids in a nascent polypeptide. A gene may also includes other regions or sequences including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotides) or at distant sites (more than 10,000 nucleotides). These non-coding sequences influence the level or rate of transcription or translation of the gene. Covalent modification of a gene may influence the rate of transcription (e.g., methylation of genomic DNA), the stability of mRNA (e.g., length of the 3′polyadenosine tail), rate of translation (e.g., 5′cap), nucleic acid repair, nuclear transport, and immunogenicity.

The term naked polynucleotide indicate that the polynucleotide is not associated with a transfection reagent or other delivery vehicle that is required for the nucleic acid or polynucleotide to be delivered to the cell. A transfection reagent is a compound or compounds that bind(s) to or complex(es) with oligonucleotides and polynucleotides, and mediates their entry into cells. The transfection reagent also mediates the binding and internalization of oligonucleotides and polynucleotides into cells. Examples of transfection reagents include, but are not limited to, cationic lipids and liposomes, polyamines, calcium phosphate precipitates, histone proteins, polyethylenimine, and polylysine complexes. It has been shown that cationic proteins like histones and protamines, or synthetic cationic polymers like polylysine, polyarginine, polyornithine, DEAE dextran, polybrene, and polyethylenimine may be effective intracellular delivery agents. Typically, the transfection reagent has a net positive charge that binds to the oligonucleotide's or polynucleotide's negative charge. The transfection reagent mediates binding of oligonucleotides and polynucleotides to cells via the reagents'positive charge (that binds to the cell membrane's negative charge) or via cell targeting signals that bind to receptors on or in the cell. For example, cationic liposomes or polylysine complexes have net positive charges that enable them to bind to DNA or RNA. Polyethylenimine, which facilitates gene transfer without additional treatments, probably disrupts endosomal function itself.

The term vector has been used in the art to describe both polynucleotides (e.g., plasmid or expression vector) and polynucleotide delivery systems (e.g., viral vector or non-viral vector). As used herein, non-viral vectors include naked polynucleotides and polynucleotide complexes including protein and polymer complexes (polyplexes), lipids and liposomes (lipoplexes), combinations of polymers and lipids (lipopolyplexes), and multilayered and recharged particles. The term non-viral vector can encompass polynucleotides with virus-derived nucleic acid sequences. The term also encompasses a polynucleotide complexed with or associated with a component of a virus, but does not include a formulation consisting of a polynucleotide contained within a viral particle or virion, or a polynucleotide complexed with or associated with an intact viral coat or capsid or an intact viral envelope. A non-viral vector is not assembled within a cell as an intact virus.

Condensing a polynucleotide means decreasing the volume that the polymer occupies. An example of condensing nucleic acid is the condensation of DNA that occurs in cells. The DNA from a human cell is approximately one meter in length but is condensed to fit in a cell nucleus that has a diameter of approximately 10 microns. The cells condense (or compact) DNA by a series of packaging mechanisms involving histones and other chromosomal proteins to form nucleosomes and chromatin. The DNA within these structures is rendered partially resistant to nuclease (DNase) action. The process of condensing polynucleotides can be used for delivering them into cells of an organism.

Two molecules are combined to form a complex—through a process called complexation or complex formation—if they are in contact with one another through noncovalent interactions such as electrostatic interactions, hydrogen bonding interactions, and hydrophobic interactions. An interpolyelectrolyte complex is a noncovalent interaction between polyelectrolytes of opposite charge.

Delivery of a polynucleotide means to transfer the polynucleotide from a container outside a vertebrate to near or within the outer cell membrane of a cell in the vertebrate. The term transfection is used herein, in general, as a substitute for the term delivery, or, more specifically, the transfer of a polynucleotide from directly outside a cell membrane to within the cell membrane. If the polynucleotide is a DNA or cDNA, it enters the nucleus where it is transcribed into a messenger RNA that is then transported into the cytoplasm where it is translated into a protein. If the nucleic acid is an mRNA transcript, it is translated in the cytoplasm by a ribosome to produce a protein. If the nucleic acid is an anti-sense nucleic acid it can interfere with DNA or RNA function in either the nucleus or cytoplasm.

A polyclonal antibody is prepared by immunizing an animal with an immunogenic composition in accordance with the present invention and collecting antisera from that immunized animal. A wide range of animal species can be used for the production of antisera. Antibodies may then be purified from the sera if desired. Typically the animal used for production of anti-antisera is selected from the group comprising: rabbit, mouse, rat, hamster, guinea pig, chicken, donkey, horse, and goat.

Genetically immunized animals may be used to produce monoclonal antibodies. The means for preparing and characterizing antibodies are well known in the art (See, e.g., Antibodies: A Laboratory Manual, Cold Spring Harbor Laboratory, 1988; and as those exemplified in U.S. Pat. No. 4,196,265, incorporated herein by reference). Following immunization, somatic cells with the potential for producing antibodies, specifically B lymphocytes (B cells), are selected for use in the mAb generating protocol. These cells may be obtained from spleens, tonsils or lymph nodes, or from a peripheral blood sample. Often, a panel of animals will have been immunized and B lymphocytes are obtained from the animal with the highest antibody titer. The antibody-producing B lymphocytes from the immunized animal are then immortalized (e.g., by retroviral transduction with ABL-Myc) or fused with cells of an immortal myeloma cell, generally one of the same species as the animal that was immunized. Any one of a number of myeloma cells may be used, as are known to those of skill in the art. Alternatively, T cell clones can be generated.

Some antigens are only weekly immunogenic or do not engage the immune system sufficiently. For these and other reasons, it is sometimes desirable to boost or enhance the body's immune response. In these instances, genetic vaccination can be enhanced by administering biological factors or chemical adjuvants to help boost immune response.

Chemical adjuvants are additions to vaccines that help boost the response to the antigen. Adjuvants are derived from a variety of sources and can be isolated from animals, plants, or are synthetic chemical compounds. An adjuvant is a compound that, when used in combination with an antigen, can augment or otherwise alter or modify the resultant immune responses. The present invention contemplates immunization with or without adjuvant. For immunization with an adjuvant, the invention is not limited to any particular type of adjuvant. Adjuvants may be used either separately or in combination. Adjuvants known in the art may be selected from the list comprising: complete Freund's adjuvant (a non-specific stimulator of the immune response containing killed Mycobacterium tuberculosis), incomplete Freund's adjuvant, agar beads, aluminum hydroxide, aluminum phosphate (alum), Quil A adjuvant (commercially available from Accurate Chemical and Scientific Corporation), QS-21 (a chemical derived from the soapbark tree), Gerbu adjuvant (commercially available from C.C. Biotech Corp.), keyhole limpet hemocyanin (KLH, derived from shell-dwelling sea animals), bacterin (i.e., killed preparations of bacterial cells), and factors normally produced to influence immune function (cytokines). Some examples of cytokines used to enhance vaccines are granulocyte/macrophage colony stimulating factor (GM-CSF, or sargramostim), the interleukins (especially IL-2 and other gamma-c interleukins), the interferons (INFs), and tumor necrosis factor alpha (TNFα). Protein-based adjuvants can be delivered as isolated proteins or as polynucleotides. Polynucleotides encoding the protein-based adjuvant are delivered to cells in a manner similar to the genetic vaccine and use the cellular machinery to produce the adjuvant.

The immune response elicited by expressed antigens can be augmented or modulated by co-expression or administration of interleukins, cytokines, interferons, growth and differentiating factors, or specific cell surface-receptor ligands. These factors can promote humoral or cell-mediated response through mobilization, activation, repression, proliferation, or maturation of immune cells or effector cells, including T cells, Thl helper T cells (which participate in cell-mediated immunity), Th2 helper T cells (which provide help for B cells), B cells, NK cells and professional antigen presenting cells such as dendritic cells (DCs). Factors such as IL-2 or IL-7 and Thl-biasing cytokines such as IFN-γ and IL-12 have been demonstrated to selectively enhance the induction of CTL-mediated immunity in mice. Alternatively, a diminished CTL responsiveness and an enhanced antigen-specific humoral response are observed with the co-delivery of Th2-biasing cytokines IL-4, IL-5, and IL-10 (Xiang Z et al. 1995; Chow YH et al. 1998; Iwasaki A et al. 1997; Kim JJ et al. 1997). Recent investigations have shown that DCs play a central role in the stimulation of cellular and humoral immunity following genetic immunization. Genetic vaccine strategies involving co-delivery of Flt3-L or GM-CSF pDNA have shown significant increases in antigen-specific antibody generation and CTL-mediated immune response (Sailaja G et al. 2003; Rakhmilevich AL et al. 2001; Sun X et al. 2002). CD154 (CD40 ligand) treatment, which promotes DC maturation, with genetic immunization has demonstrated enhancement of both humoral and cellular antigen-specific immunological response to antigens like HIV-1 encoded proteins (Ihata A et al. 1999). It is readily conceivable that prior treatment with certain stimulators will prime the host for a subsequent antigen delivery, and thus result in a stronger or more rapid antigen-specific immune response.

Several means to enhance the immune response generated by genetic immunization are readily conceivable. For example, genes of compounds may be delivered to cells which increase the number of histocompatibility antigens on the cell surfaces. Polynucleotide delivery can also be combined with an agent to stimulate cytokine production or release, causing lymphocyte or other immune cell proliferation or activation. Immunomodulators, such as cytokines, including interferons and interleukins, or polynucleotides expressing immunomodulators may also be delivered to the animal. The polynucleotide itself may also be covalently modified with a compound to enhance an immune response. Also, the polynucleotide associated with a ligand that directs it to a specific cell type.

Combinations of immunomodulators may be used in accordance with the present invention. In addition, relative timing of administration of an immunomodulator may be important for maximal immune response or for eliciting the desired type of immune response.

Another method for increasing antibody induction is by formation of multimeric antigens, which can stimulate B cells without T help. This can be achieved by generating fusion of the antigen with pentraxin proteins (e.g., C reactive protein, serum amyloid protein) or IgM, which form pentamers. A similar approach was recently described to significantly enhance genetic immunization antibody induction using a cartilage oligomeric matrix protein sequence.

It is possible to use the expression vector used to immunize the animal to also prepare antigen to test the titer of the resultant immune response. The expression vector used for genetic immunization can be transfected into culture cells using methods standard in the art. Following transfection, the cells express the transgene and can therefore be used as a source of material to screen for antibodies. The cells can be used in situ (e.g., immunohistochemistry, flow cytometry) or extracts can be made for use in other immunological assays (e.g., Western blotting, ELISA).

### EXAMPLES

The following examples are provided in order to demonstrate and further illustrate certain preferred embodiments and aspects of the present invention and are not to be construed as limiting the scope thereof.

### Example 1

Hydrodynamic Tail Vein (HTV) Delivery.

Hydrodynamic intravascular injection has been shown to be a reliable and efficient method of functionally delivering polynucleotides to a broad distribution of cells in vivo, U.S. Pat. No. 6,265,387 (incorporated herein by reference). Hydrodynamic tail vein injection is described in U.S. Pat. No. 6,627,616 (incorporated herein by reference). For hydrodynamic tail vein delivery in mouse, plasmid DNA was diluted in an injection solution volume equal to 1 ml per 10 grams body weight and injected into the lateral tail vein in 6 to 7 seconds. The maximum volume delivered was capped at 3.0 ml (mice ≧30 gram body weight). A similar procedure was used for hydrodynamic tail vein delivery in rats, with a delivery time of 18-22 seconds and a maximum volume of 20 ml (rats ≧200 g).

### Example 2

Hydrodynamic Limb Vein (HLV) Delivery.

Intravenous pDNA injection into veins of limbs temporarily isolated by a tourniquet resulted in very high gene transfer to skeletal muscle cells. This hydrodynamic limb vein procedure is readily applied in small rodents (mouse, rat and rabbit) and larger mammals (dog and primate) with similar transfection efficiencies. For immunizations, six-week old ICR mice (˜20 g), six-to-seven week old (˜125 g) Sprague-Dawley rats and two-month old (˜2.2 kg) New Zealand White rabbits were used. Hydrodynamic limb vein gene delivery was performed as described in US-2004-0242528(incorporated herein by reference). Hydrodynamic arterial injection for delivery to limb skeletal muscle can also be performed as described in U.S. patent application No. 09/707,000 (incorporated herein by reference). For the following examples, a tourniquet was wrapped around the upper hind limb just above the quadriceps and tightened into place with a hemostat to block blood flow to and from the leg. A small incision was made to expose the distal great (or medial) saphenous vein. A catheter was inserted into the vein. For mouse injections, a 30 gauge needle catheter was inserted and advanced so that the tip of the needle was positioned just above the knee in an antegrade orientation. A syringe pump was used to deliver the polynucleotide containing saline solution (0.05 ml/g in mice; 0.08 ml/g in rats; 33 ml/kg in rabbits) at a flow rate of about 4.5 ml/min. For some injections, an efflux enhancer solution containing (0.017% papaverine in 0.25 ml saline) was injected into the limb at a flow rate of 4.5 ml/min 1-5 min. prior to the polynucleotide containing solution. The solution(s) were injected in the direction of normal blood flow through the vein. The needle was retracted and the tourniquet released two minutes after pDNA delivery. Bleeding was controlled with pressure and a hemostatic sponge. The incisions were closed with 4-0 Vicryl sutures.

For direct intramuscular injections in mice, 50 μg pDNA was injected in 100 μl saline solution into the quadriceps, using a 30 gauge needle in 2-3 seconds.

### Example 3

Genetic Immunization by Hydrodynamic Tail Vein Plasmid DNA Delivery.

A. Mice:

1) Luciferase Antigen. Mice were injected 5 times via HVT injection with 50 μg pCI-Luc (delivered on days 0, 14, 21, 28 and 35). Sera were collected on day 35 and 42 and assayed for anti-luciferase Ab by ELISA. On day 35, the average level of anti-luciferase Abs in HTV immunized mice was 334.3 ±122.2 μg/ml (5 mice). Antibody levels approximately doubled by day 42. All 5 mice exhibited an immune response as determined by antigen-specific antibody production.

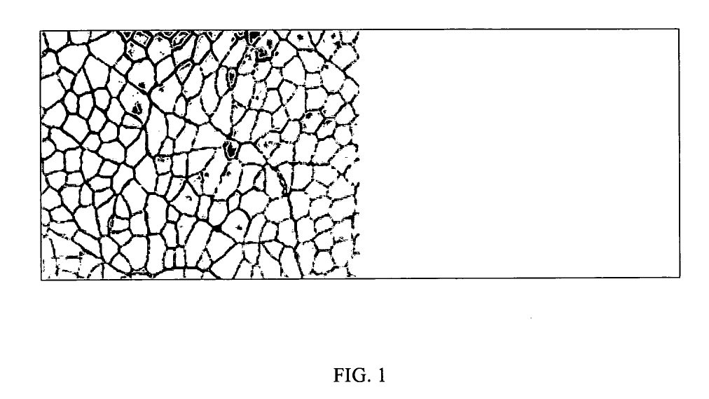

2) Human dystrophin. An anti-human dystrophin antibody was generated in ICR mice by hydrodynamic tail vein genetic immunization. The mice were primed and boosted by hydrodynamic tail vein delivery of 100 μg of a human dystrophin expression cassette (two boosts at 2 and 3 weeks after the prime). Sera were obtained 3 days after the second boost and used to stain for human dystrophin expression in mdx (dystrophin deficient) mice previously injected with 10 μg of the same expression vector (IM). Immunohistochemistry with the antisera showed the presence of myofibers expressing human dystrophin in a typical dystrophin staining pattern. The staining pattern was identical to that obtained with commercially available anti-human dystrophin antibodies. Thus, intravascular genetic immunization can result in the generation of antibodies against clinically relevant target proteins with titers sufficient to be used for immunohistochemistry. The antisera was further shown to cross react with the mouse dystrophin in ICR (dystrophin positive mice). Dystrophin staining in ICR mouse with the antisera is shown in FIG. 1. The left panel of FIG. 1 shows mouse skeletal muscle stained with the anti-human dystrophin polyclonal antisera using a labeled anti-mouse IgG secondary antibody for fluorescence detection. The right panel shows mouse skeletal muscle stained with a commercially available anti-human dystrophin monoclonal antibody that does not cross react with mouse dystrophin.

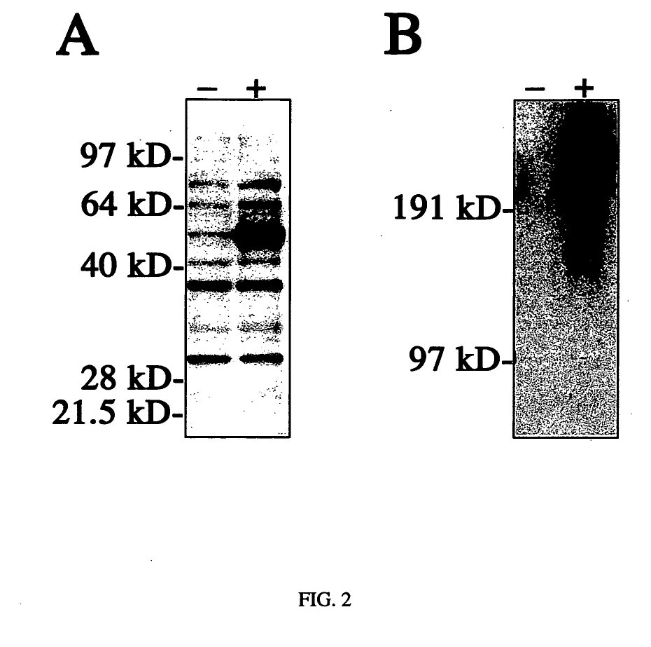

3) Human CD4 and canine dystrophin. Mice were immunized via HTV injection with polynucleotides encoding either a truncated human CD 4 protein or canine dystrophin. CD4 is a membrane-bound antigen and dystrophin is an intracellular antigen. 50 μg plasmid DNA was injected into the tail vein of mice on days 0, 14, 21 and 28. Blots, containing extracts from cells expressing either the immunizing antigen (+lanes) or a control protein (− lanes) were probed with sera sampled on day 35. Sera were diluted 1:100. The CD4 protein has a predicted protein size of approximately 46 kD and the canine dystrophin has a predicted protein size of approximately 425 kD. FIG. 2 shows that each of the mice produced antigen-specific antibodies. The experiment was repeated with similar results: all HTV injected animals developed antibodies to the protein encoded by the injected nucleic acid. In 25 mice injected with hCD4 expression vector, all 25 mice generated anti-hCD4 specific antibodies.

4) Mouse immunization success rate. Of 21 different antigens, HTV genetic immunization induced antibody production against 14 of the antigens (6 or more mice per antigen). For the 14 antigens that induced antibody production, the immune response was observed in 272 out of 276 mice (including 25 out of 25 mice immunized with hCD4 and 189 out of 189 mice immunized with Luciferase). Only antibody production was measured. For animals in which no antibodies were detected by ELISA or Western blot analyses, no other tests were performed to detect an immune response, though other forms of immune response were possible.

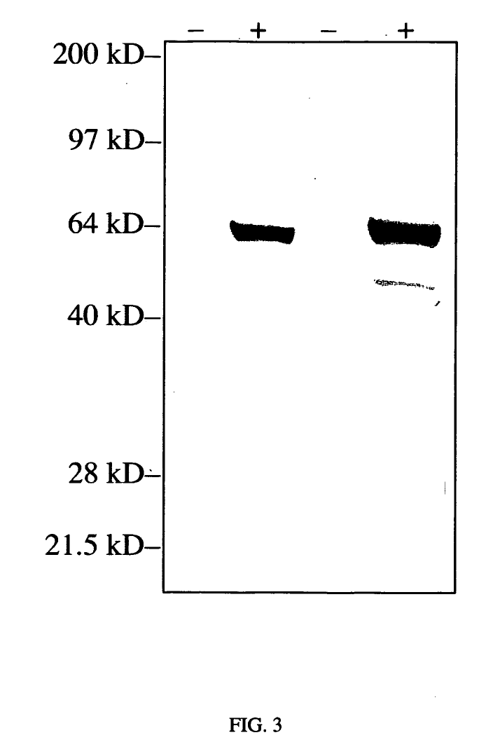

B. Rats. Rats were genetically immunized via intravascular delivery of polynucleotide as described for mouse immunization. 500 μg pMIR48 in 20 ml was injected into the tail vein of rats in 20 sec. Rats were injected on days 0, 14, 21 and 28. On day 35 animals were bled and the sera were tested for the presence of anti-luciferase antibodies by Western blot. The data in FIG. 3 shows luciferase-specific antibodies were present in the injected rats (−lane =cell extract lacking antigen; +lane =cell extract containing antigen), demonstrating the application of intravascular genetic immunization in larger rodents. Four of four rats immunized in this way produced antigen-specific antibodies.

### Example 5

Single Injection Genetic Immunization.

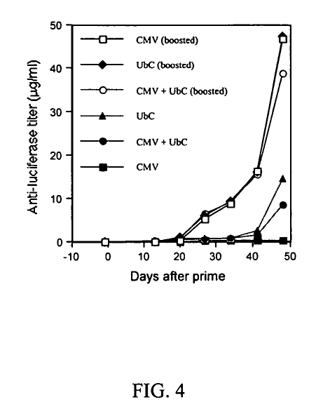

Sustained expression of the antigen may requires fewer boost injections. Genetic immunization using two luciferase vectors: pMIR48 (CMV promoter) and pMIR68 (ubiquitin C promoter) were compared. We have shown that pMIR68 generates stable luciferase expression for many months at a level about ten-fold below CMV-driven peak levels. FIG. 4 shows the result of antibody induction following hydrodynamic tail vein delivery of 10 μg pMIR48 or pMIR68 alone or a combination of the two (5 μg each). We also compared the effect of 2 boosts versus no boost. A single injection of pMIR48 did not result in significant antibody titers by day 42, whereas pMIR68 immunized mice showed significant levels of anti-luciferase antibodies with a single injection. Boosting increased antibody levels significantly, and no differences between the different plasmid DNA groups were observed.

### Example 6

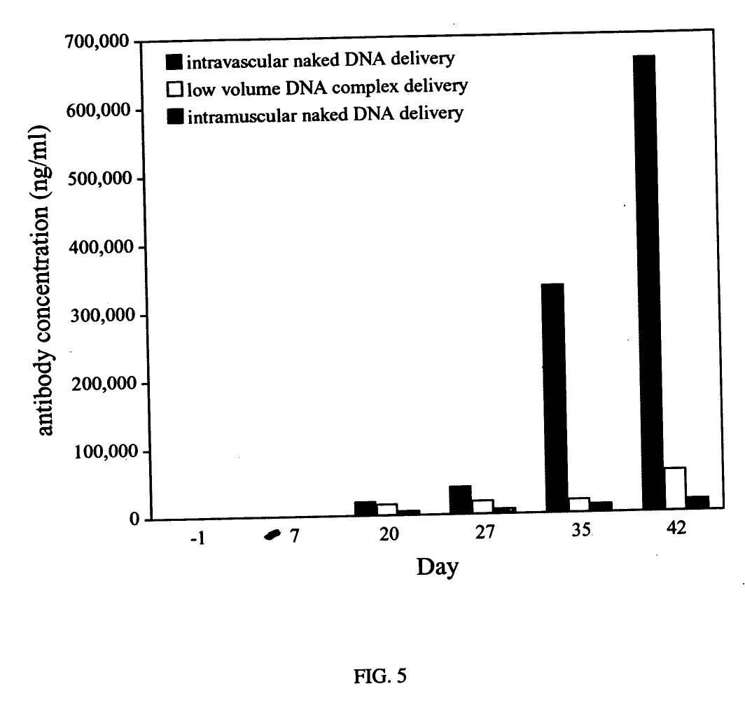

Genetic Immunization of Mice: Comparison of Hydrodynamic Tail Vein Injection vs. Direct Intramuscular Injection. A. The luciferase expression vector pMIR48 was administered to ICR mice by each of three methods: intramuscular and intravascular delivery of naked pDNA, and intravascular delivery of pDNA particles (5 animal per group). For direct intramuscular injections in the quadriceps, 50 μg plasmid DNA in 100 μl saline was injected. For intravascular delivery via hydrodynamic tail vein injection, 50 μg plasmid DNA in 1 ml Ringer's solution per 10 g mouse body weight was injected in about 7 seconds. For low-pressure tail vein injection, 50 μg plasmid DNA was complexed with the polycation polyethylenimine (PEI) and recharged with the polyanion polyacrylic acid (PAA) at a ratio of 1:6:1 (wt:wt:wt) in a volume of 50 μl. Mice were injected on days 0, 14, 21 and 28. To quantitate anti-luciferase antibody titers, sequential serum samples were taken before the initial (prime) injection and 7 days after each injection and analyzed by standard ELISA test. A standard curve was generated using a commercially available anti-luciferase antibody. The results (FIG. 5) demonstrated that increased pressure intravascular delivery of naked pDNA resulted in higher titers and more rapid induction of anti-luciferase antibodies than the more conventional injection into skeletal muscle. Dose response experiments (not shown) have indicated that after two booster injections with 10 μg pDNA delivered IV resulted in higher titers than the highest dose delivered IM (100 μg). PEI/PAA particles are better than IM injection, even though the final titers are lower than after IV immunization. Of the mice immunized by intravascular tail vein injection of DNA, all animals developed an immune response.

B. 50 or 100 μg of DNA encoding firefly luciferase were injected into mice 4 times as described above. The first injection, the prime injection, occurred at day 0. Subsequent injections occurred on days 14, 21 and 28. Antisera from mice were tested at various times before, during and after immunization. As shown in Table 1, genetic immunization by intravascular delivery of polynucleotide resulted in higher antigen-specific antibody titers than did intramuscular injection of polynucleotide. Similar results were observed in animals which received injections in which the DNA was in: a) standard Ringers'solution, b) standard Ringers'solution +5% mannitol or c) 50% standard Ringers'solution/50% saline +3.75% mannitol. Levels of anti-luciferase antibody titers (μg/ml antibody concentration) generated by intravascular tail vein injection versus direct intramuscular injection. Levels shown are averages of five mice per group. All animals immunized by hydrodynamic tail vein injection responded and developed an anti-luciferase antibodies.

TABLE 1

| | Intravascular | | Intramuscular | |
|---|---|---|---|---|
| day | 50 μg DNA | 100 μg DNA | 50 μg DNA | 100 μg DNA |
| 0 | 0.05 ± 0.03 | 0.04 ± 0.01 | 0.09 ± 0.05 | 0.04 ± 0.01 |
| 7 | 0.12 ± 0.04 | 0.23 ± 0.12 | 0.07 ± 0.02 | 0.06 ± 0.01 |
| 20 | 17.7 ± 20.3 | 20.3 ± 15.6 | 1.06 ± 1.70 | 1.85 ± 2.80 |
| 27 | 36.8 ± 25.9 | 79.2 ± 28.9 | 1.90 ± 2.42 | 5.98 ± 4.25 |
| 35 | 334 ± 244 | 344 ± 234 | 6.62 ± 9.57 | 3.73 ± 4.51 |
| 42 | 668 ± 366 | 926 ± 229 | 14.3 ± 21.8 | 17.4 ± 16.8 |

### Example 7

Genetic Immunization by Hydrodynamic Limb Vein Plasmid DNA Delivery.

A. Mice.

1) Luciferase antigen. Mice were injected with 1, 2, 3 or 4 doses of pCI-Luc. Groups of mice received repeat doses of pCI-Luc in the same limb or in alternate limbs. ELISA results demonstrated that very high levels of anti-luciferase Abs were generated with only two HLV gene deliveries in all mice tested. Injection into a single limb or to alternating limbs resulted in similar Ab levels. As controls, two mice were immunized via plasmid delivery to the liver using tail vein injections (retrograde injection). Mice received injections on the same day as indicated above. For the tail vein injections, 10 μg plasmid DNA in 2.5 ml Ringer's solution per injection was injected into the tail vein using a 27 gauge needle. The entire volume was delivered in less than 10 sec.

To monitor induction of an immune reaction to luciferase, the animals were bled on days 0, 13, 20, 27, 34, 41 and 48. The blood was allowed to clot and the sample was centrifuged to recover the sera. Sera were analyzed for the presence of antibodies to luciferase using an ELISA, as follows: 96-well plates were coated with a recombinant luciferase protein (Promega, Madison, WI) by incubation of 100 μl of 2 μg/ml protein in 0.1 M carbonated buffer per well. Plates were incubated overnight at 4° C., then washed three times with PBS containing 0.05% Tween 20. Wells are blocked with 200 μl PBS +1% non-fat dried milk for 1.5 h at RT and washed three times as above. Mouse sera were diluted in PBS +1% milk. 100 μgl diluted sera were added to wells in duplicate and incubated 1.5 h at RT. The plates were washed three times as above. 100 μl anti-mouse polyvalent antibody conjugated to horseradish peroxidase (Sigma, St. Louis, Mo.) diluted 1:20,000 in the PBS +1% milk buffer was added to each well. The plates are washed five times as above. 100 μl tetramethyl-benzidine (Sigma) was added to each well and the samples were allowed to develop. The reaction was stopped by addition of 100 μl 1.0 M H₂SO₄ per well and the absorbance was read at 450 nm. A standard curve was generated using a goat anti-luciferase horseradish peroxidase conjugate (Sigma). The results are shown in Table 2. The presence of anti-luciferase antibodies in the mouse sera indicates successful induction of an immune response.

TABLE 2
Antibody concentration (μg/ml) in mice genetically immunized
via injection of plasmid DNA into either tail vein or saphenous
vein. All mice injected, tail vein injection and saphenous
vein injection, developed an immune response.

| | | saphenous |
|---|---|---|
| day | tail vein | vein |
| 0 | 0.13 | 0.09 |
| 13 | 0.06 | 2.03 |
| 20 | 1.72 | 51.6 |
| 27 | 47.1 | 175 |
| 34 | 106 | 471 |
| 41 | 174 | 332 |
| 48 | 235 | 393 |

2) Hepatitis B virus e antigen. Mice were injected via HLV injection four times (day 0, 14, 21 and 28) with 50 μg of pDNA vectors expressing the. secreted protein hepatitis B virus e antigen (HBVe). Western blot analysis of HBVe using injected mouse antisera showed detection of the unmodified C antigen precursor (24 kDa) and the mature e antigen (16 kDa), data not shown.

2) Rat erythropoietin antigen. Mice were injected via HLV injection four times (day 0, 14, 21 and 28) with 50 μg of pDNA vectors expressing the secreted protein rat erythropoietin (rEPO). Western blot analysis of rEPO using injected mouse antisera showed detection of the protein of predicted size, 34 kDa, data not shown.

3) Mouse immunization success rate. Mice immunized via HLV plasmid DNA injection generated specific, high titer Abs to 83% of expressed proteins of tested antigens (5 of 6 antigens; luciferase, human gplOO, rEPO, HBVe, hVEGF165). Thus, HLV gene delivery is an effective method for generating Abs to expressed proteins in mice. For antigens which generated an antibody production, all mice injected developed an immune response. The presence of immune responses other than antibody production was not tested. For three antigens for which no antibody production was detected following immunization via tail vein injection, antibody production was observed when the same plasmid was delivered by limb vein injection.

Three of the antigens that failed to elicit antibody production following HTV genetic immunization were predicted to be secreted proteins. For these three antigens, HLV genetic immunization was successful in eliciting antibody production. Thus, the site of expression may influence the properties of the antigen in generating an antibody production immune response.

B. Rats. Rats were injected 4 times with 500 μg of the luciferase expression vector Sera were collected at several time points after immunization and analyzed for anti-luciferase Abs by ELISA. All rats injected produced luciferase antigen-specific antibodies.

Four ˜150 g Sprague-Dawley rats per group were immunized with 500 μg pCI-Luc (day 0, 14, 21, 28). Group 1 animals were immunized by delivery of antigen-encoding polynucleotide via saphenous vein injection Plasmid DNA in 3 ml of normal saline solution (NSS) was used for each injection. Blood flow to and from the limb was restricted just prior to and during the injection, and for 2 min post-injection by placing a tourniquet around the upper leg (μOust proximal to/or partially over the quadriceps muscle group). The solution was injected into the great saphenous vein of the distal hind limb at a rate of 3 ml per ˜20 seconds (10 ml/min). The intravenous injections were performed in an anterograde direction (i.e., with the blood flow) via a needle catheter connected to a programmable Harvard PHD 2000 syringe pump (Harvard Instruments). Group 2 animals received immunization via hydrodynamic delivery of polynucleotide through the tail vein. Immunizations occurred on days 0, 13, 20, 20, 27, 25 and 42 and animals were bled on days 7, 20 and 28. Sera were separated and tested in a single ELISA (Table 3).

TABLE 3
Results are shown in μg/ml antibody concentration.

| Day | Tail vein | Saphenous vein |
|---|---|---|
| 0 | 0.15 ± 0.10 | 0.34 ± 0.28 |
| 13 | 0.26 ± 0.11 | 13.3 ± 19.8 |
| 20 | 0.31 ± 0.13 | 62.9 ± 75.3 |
| 27 | 0.42 ± 0.07 | 102 ± 102 |
| 35 | 0.75 ± 0.53 | 469 ± 308 |
| 42 | 0.61 ± 0.27 | 490 ± 370 |

Similar results were obtained with two other antigens.

C. Rabbits. Rabbits were injected with 1 mg/kg body weight pCI-Luc, either 2 (day 0, 14) or 3 times (day 0, 14, 21), via the hind limb saphenous vein (two rabbits per group). Individual rabbit anti-luciferase Ab levels were measured by ELISA on days 35 and 42. High levels of anti-luciferase Abs were present in the sera on day 35 after either two injections (100.4 and 585.0 μg/ml) or three injections (286.8 and 345.7 μg/ml). Titers were approximately double on day 42.

Four rabbits were injected on days 0, 14, 21 and 28 with a plasmids encoding the firefly luciferase gene under control of the cytomegalovirus promoter (pMIR48) and the ubiquitin C promoter and a hepatic control region for enhancement of long-term expression (pMIR68). Two animals also received a plasmid encoding murine interleukin 2 under control of the cytomegalovirus promoter (PMIR 152).

For each injection, a solution containing the plasmid was inserted into the lumen of the saphenous vein as follows: A latex tourniquet was wrapped around the upper hind limb to block blood flow into and out of the leg and tightened into place with a hemostat. Injections were done into either the great or the small saphenous vein. A 23 gauge catheter was inserted, in antegrade orientation, into the lumen of the vein. A syringe pump was used to inject an efflux enhancer solution (1.0 mg papaverine in 6 ml) at a flow rate of 4-5 ml/min. One to five minutes later a solution containing plasmid DNA was injected through the catheter (1 mg/kg pMIR48 or pMIR68; 2 mg/kg pMIR152 in 18-44 ml saline, 14 ml/kg animal weight.) The solution was injected in 18-30 seconds (1-2 ml/sec). The volume of solution and rate of injection were varied depending on the weight of the rabbit. The solution was injected in the direction of normal blood flow through the vein. The tourniquet was removed two minutes after the injection. Bleeding from the incision and vein puncture was controlled with pressure and a hemostatic sponge. The incision was closed with 4-0 Braunamid suture. The procedure was completed in ˜20 min.

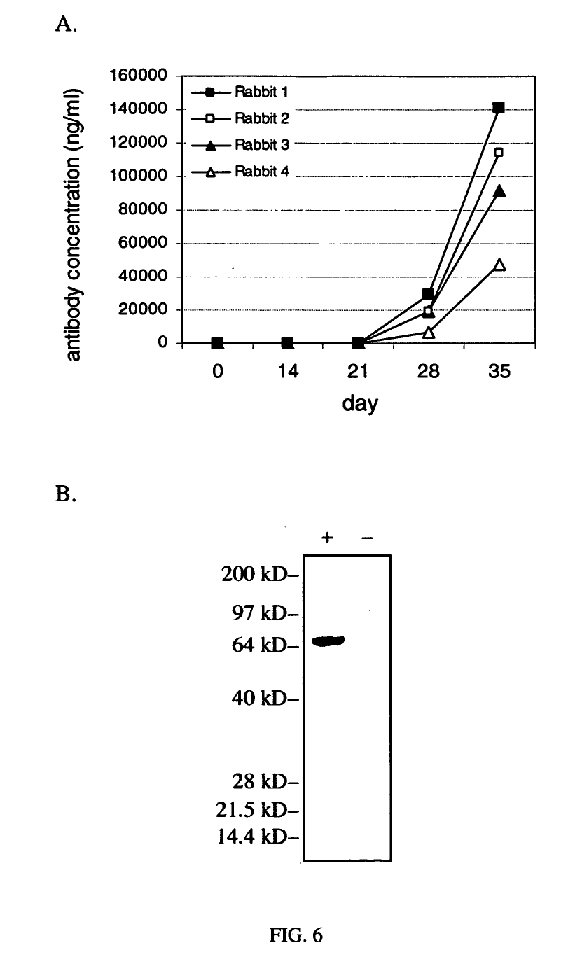

To monitor induction of an immune reaction to luciferase in the animals, animals were bled via the ear vein. The presence of antibodies in the sera, indicating induction of an immune response, was determined by ELISA and Western blot. The results are shown in FIG. 6. The presence of anti-luciferase antibodies in the rabbit sera indicates successful induction of an immune response. These results demonstrate the applicability of intravascular genetic immunization in larger animals that can be used to produce polyclonal antibodies on a larger scale. 100% of rabbits (16 out of 16) injected by HLV gene delivery injection generated high titer, specific Abs.

### Example 8

Antibodies Generated via Intravascular Genetic Immunization Maintain High Titers over Long-term.

Four mice were immunized via intravascular tail vein delivery of polynucleotides as described above. Mice were injected with 10 μg pMIR48 on days 0, 14, 21 and 28. High titer was observed in all four mice at day 48 as tested by ELISA, three weeks after the last boost. This level was maintained for at least another 32 days (Table 4).

TABLE 4

| | anti-luciferase antibody titer |
|---|---|
| day | (μg Ab/ml serum) |
| 0 | 0.01 |
| 13 | 0.02 |
| 20 | 0.39 |
| 27 | 5.40 |
| 34 | 8.76 |
| 41 | 16.5 |
| 48 | 46.9 |
| 76 | 48.5 |

### Example 9

Generation of Antigen in Vitro for Screening Antibodies.

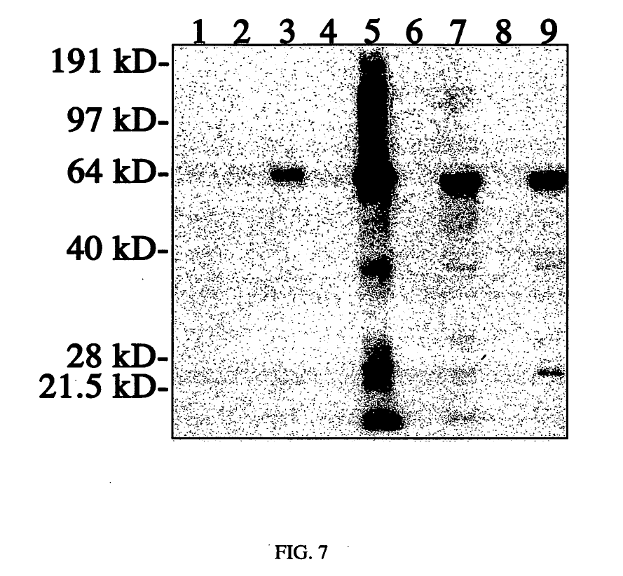

COS-7, HEK 293 and Hepa-lclc7 cells were transfected with pCI-LacZ or pCI-Luc vectors. The cells were transfected with 2 μg pDNA per 35-mm well using TRANSIT transfection reagents (Mirus Bio Corporation, Madison, WI). Cells were washed twice with PBS, and resuspended in sample buffer (10⁷ cells/ml). Extracts (5×10⁴ cells) were separated on NuPAGE Tris-Acetate gels and blotted onto Hybond-P membranes. The membranes were incubated with a 1:5,000 dilution of sera pooled from 4 mice immunized with pCI-Luc (HTV injection). For immunization, mice were injected on days 0, 14, 21, 28 and 35. Following antibody binding, the blots were washed and incubated with a HRP-labeled goat-anti-mouse IgG (1:5000 dilution). Specific binding was detected by chemiluminescent development (FIG. 7). A single protein was detected only in the lanes loaded with luciferase transfected cells. Thus, in vitro transfection can generate sufficient amounts of antigen to allow for screening.

### Example 10

Hybridoma Fusion using Splenocytes from Mice Immunized via HTV Injection of Plasmid DNA.

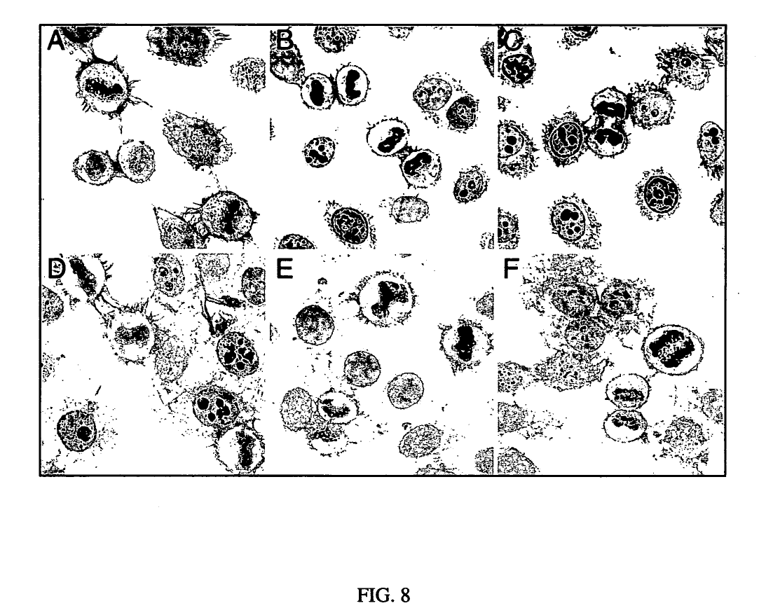

Six mice were immunized with pMIR167 encoding human Ki67, a chromatin-binding protein, via four injections into tail vein as described above. Analysis of the mouse antisera showed very strong signal (results not shown). Animals were given a fifth immunization on day 105 and spleens were harvested four days later. Splenocytes were frozen and processed for hybridoma fusion using methods standard in the art. 46 clones were isolated that presented typical Ki-67 pattern in immuno-cytochemical staining. None of the supernatants cross-reacted with mouse ki67 protein. Two cross-reacted with rat ki67 protein. Almost all cross-reacted with monkey Ki67 protein. Five of these culture supernatants, along with a commercially available anti-Ki67 antibody are shown detecting Ki67 in HeLa cells in FIG. 8. All six animals injected yielded antibody producing immune cells.

### Example 11

Enhanced Immune Response by Co-delivery of Cytokine Expression Vectors.

The immunomodulator Flt3-Ligand (Flt3-L) acts on CD34+ progenitor cells and results in increases in DC and NK cells. Intravascular delivery of a CMV promoter-driven Flt3-L vector into ICR mice via tail vein injection was performed to determine the effects of delivery of the Flt3-L gene. Different levels of the expression vector were injected and the number and composition of spleen cells was analyzed after 10 days. Delivery of 10 μg murine Flt3-Ligand pDNA increased the total splenocyte count 3.8 fold (260 million cells per spleen for Flt3-L treated mice compared to 68 million cells per spleen for control mice). Furthermore, the splenocytes demonstrated an increase in the percentage of CDllc+ dendritic cells. 2.3% CDllc+ splenocytes were observed in control mice while 24.5% CDllc+ splenocytes were observed in mice receiving Flt3-L pDNA. A dose-dependent response in total number of splenocytes and CDllc+ cells was observed when delivering a range of 1-50 μg/mouse of Flt3-L pDNA.

### Example 12

Intravascular Injection of Non-viral DNA Particles.

ICR mice were immunized with pCI-Luc, an expression vector in which luciferase is under transcriptional control of the human CMV promoter. Several gene transfer routes were tested: intravascular delivery of plasmid DNA into the tail vein, intravascular delivery into the tail vein of plasmid DNA complexed with linear polyethylenimine (IPEI) and polyacrylamide (PAA), and direct IM injection of naked plasmid DNA (3-5 mice per group). All mice were boosted on day 21 using the same gene delivery method as used for the prime. Immunization using intravascular delivery resulted in a strong antibody response (see Table 5). For instance, one day following intravascular pCI-Luc delivery, luciferase expression in the liver averages 5 μg of protein. PEI/PAA, resulting in luciferase expression predominantly in the lungs, provides a viable alternative for genetic immunization technique. In contrast, the classic injection of plasmid DNA directly into skeletal muscle is not nearly as effective at generating an antibody response.

TABLE 5

| | | anti-luciferase antibody | |
|---|---|---|---|
| day | immunization | titer range | ng/ml average |
| 0 | Pre-immune | 10-100 | 73 |
| 21 | Post prime (HTV) | 100-10,000 | 3,050 |
| 21 | Post prime (lPEI/PAA) | 100 | Not determined |
| 35 | Post boost (HTV) | 100-100,000 | 106,075 |
| 35 | Post boost (lPEI/PAA) | 100-1,000 | >200,000 |

### Examples 13

Codon Optimization.

Many viruses such as Respiratory Syncytial Virus (RSV) and SARS CoV replicate in the cytoplasm of infected cells and use their own virally encoded polymerases and transcriptases. When genes from such viruses are expressed from mammalian expression cassettes, they are subject to the normal host nuclear processes such as polyadenylation, splicing, and RNA polymerase II mediated transcription. This may lead to incorrect or low levels of expression. Therefore, in order to produce high levels of gene product, it may be important that the sequence encoding that gene be altered to mimic a typical nuclear gene. This codon optimization entails constructing the gene using frequently used codons according to codon-usage tables for the host species and eliminating potential splicing, polyadenylation, and anti-sense start sites present in the native microbial sequence.

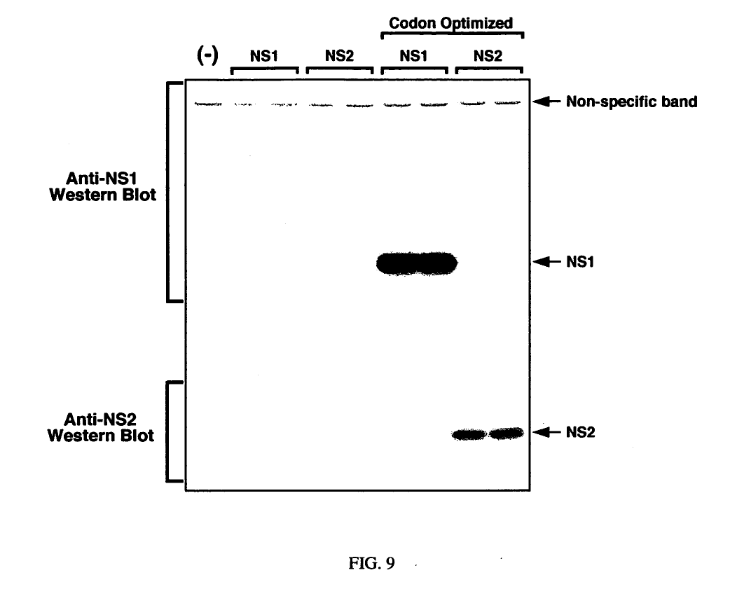

To illustrate both the utility and importance of codon optimization, the human RSV mRNAs encoding the non-structural proteins, NS1 and NS2, were cloned using standard RT-PCR from total cellular RNA made from RSV infected HEp-2 cells. The RSV ORFs were cloned into standard expression vectors downstream of the CMV immediate early promoter. The same RSV NS1 and NS2 ORFs were also codon optimized and the resulting ORFs were synthesized. These new NS1 and NS2 encoding DNA fragments were cloned into the same expression vectors. All four expression vectors have identical and optimal translational context surrounding their ATG start codons based on Kozak's rules. Each of the four NS1 and NS2 expression vectors were then transfected into HeLa cells, and total cell lysates were prepared 24 hours post-transfection for Western blotting. As illustrated in FIG. 9, there was no detectable expression of NS1 and NS2 from the expression vectors containing the non-optimized ORFs. However, transfection of the plasmids containing the optimized ORFs led to high-level expression of both NS 1 and NS2.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A method of generating antibodies specific to an antigen in an animal comprising:
a) providing a nucleic acid sequence encoding a peptide containing at least one antigenic determinant of the antigen operatively linked to one or more control sequences such that the nucleic acid sequence is capable of being expressed in a cell in the animal;
b) optionally formulating the nucleic acid sequence into a particle by complexation with one or more polymers;
c) injecting the nucleic acid sequence by hydrodynamic intravascular injection into a vessel connected to a tissue in the animal, thereby delivering the nucleic acid sequence to an extravascular cell in the tissue, expressing the nucleic acid sequence in the cell, and generating an antibody response in the animal to the expressed nucleic acid sequence; and,
d) isolating antibodies specific to the antigen from the animal.

2. The method of claim 1, wherein the extravascular cell is a lymphoid cell.

3. The method of claim 1, wherein the extravascular cell consists of a liver cell.

4. The method of claim 1, wherein the extravascular cell consists of a muscle cell.

5. The method of claim 1 wherein the vessel consists of a tail vein.

6. The method of claim 1 wherein the vessel consists of a limb vessel.

7. The method of claim 1, wherein the nucleic acid consists of DNA.

8. The method of claim 7 wherein the DNA consists of a plasmid.

9. The method of claim 1, wherein the animal consists of a rodent.

10. The method of claim 9 wherein the rodent consists of a mouse.

11. The method of claim 9 wherein the rodent consists of a rat.

12. The method of claim 9 wherein the rodent consists of a rabbit.

13. A method of generating antibodies or immune cells specific to an antigen comprising:
a) providing a non-viral nucleic acid encoding at least one antigenic determinant of the antigen;
b) injecting the non-viral nucleic acid into a rodent by hydrodynamic tail vein injection, thereby delivering the non-viral nucleic acid to a liver cell wherein the antigen is expressed and an immune response directed against the expressed antigen is induced; and,
c) isolating from the rodent the antibodies or immune cells producing the antibodies.

14. The process of claim 13 wherein the rodent consists of a mouse.

15. The process of claim 13 wherein the rodent consists of a rat.

16. The process of claim 13 wherein the immune cells consist of B lymphocytes.

17. The process of claim 16 further comprising immortalizing the B lymphocytes.

18. The process of claim 17 wherein the immortalized B lymphocytes are used to generate monoclonal antibodies.

19. The process of claim 13 wherein the immune cells consists of T lymphocytes.

20. A method of vaccinating an animal comprising:
a) providing a non-viral vector containing a gene encoding at least one antigenic determinant of an antigen to which vaccination is desired, wherein the gene is operatively linked to one or more control sequences such that the gene is capable of being expressed in a cell in the animal;
b) injecting the non-viral vector by hydrodynamic intravascular injection into a vessel connected to a tissue in the animal, thereby delivering the gene to an extravascular cell in the tissue and expressing the gene in the cell, wherein expression of the gene in the cell results in generating an immune response in the animal thereby vaccinating the animal.

21. The method of claim 20, wherein the extravascular cell consists of a liver cell.

22. The method of claim 20 wherein the vessel consists of a limb vessel.

23. The method of claim 22, wherein the extravascular cell consists of a muscle cell.

24. The method of claim 20, wherein the non-viral vector consists of naked DNA.

25. The method of claim 20 wherein the non-viral vector is selected from the lists consisting of: lipoplex, polyplex, or lipopolyplex, multilayered particle or recharged particle.

26. A method of inducing a cellular immune response to an antigen in an animal comprising:
a) providing a non-viral vector containing a gene encoding at least one antigenic determinant of the antigen, wherein the gene is operatively linked to one or more control sequences such that the gene is capable of being expressed in a cell in the animal;
b) injecting the non-viral vector by hydrodynamic intravascular injection into a vessel connected to a tissue in the animal, thereby delivering the gene to an extravascular cell in the tissue and expressing the gene in the cell, wherein expression of the gene in the cell results in inducing the cellular immune response in the animal.

27. The method of claim 26, wherein the extravascular cell consists of a liver cell.

28. The method of claim 26 wherein the vessel consists of a limb vessel.

29. The method of claim 28, wherein the extravascular cell consists of a muscle cell.

30. The method of claim 26, wherein the non-viral vector consists of naked DNA.

31. The method of claim 26 wherein the non-viral vector is selected from the lists consisting of: lipoplex, polyplex, or lipopolyplex, multilayered particle or recharged particle.
